(19) United States
(12) Patent Application Publication
(10) Pub. No.: US 2016/0221745 A1
(43) Pub. Date: Aug. 4, 2016

(54) Hair Conditioning Sunscreen Device

(71) Applicant: Christine Williams, Sarasota, FL (US)

(72) Inventor: Christine Williams, Sarasota, FL (US)

(21) Appl. No.: 14/608,583

(22) Filed: Jan. 29, 2015

(51) Int. Cl. B65D 83/44; A61K 8/40; A61K 8/27; A61K 8/35; A61K 8/49; B65D 83/20; A61K 8/67; A61K 8/92; A61K 8/65; A61Q 5/12; A61K 8/97; A61Q 17/04; A61K 8/37; A61K 8/46

(52) U.S. Cl. CPC B65D 83/44; A61K 8/37; A61K 8/40; A61K 8/27; A61K 8/35; A61K 8/4946; A61K 8/466; A61K 8/673; A61K 8/922; A61K 8/65; A61Q 5/12; A61K 8/97; A61Q 17/04; B65D 83/20; A61K 2800/87; A61K 2800/5922

(57) ABSTRACT

A hair conditioning sunscreen device for applying sunscreen and hair conditioner to a scalp covered by fine hair to provide protection to the scalp and inhibit damage to the hair includes a mixture of a sunscreen and a hair conditioner wherein the mixture is applicable to skin covered by fine hair such that the skin is protected from ultraviolet light and the hair is conditioned by the hair conditioner.

### BACKGROUND OF THE DISCLOSURE

### Field of the Disclosure

The disclosure relates to sunscreen devices and more particularly pertains to a new sunscreen device for applying sunscreen and hair conditioner to a scalp covered by fine hair to provide protection to the scalp and inhibit damage to the hair.

### SUMMARY OF THE DISCLOSURE

An embodiment of the disclosure meets the needs presented above by generally comprising a mixture of a sunscreen and a hair conditioner wherein the mixture is applicable to skin covered by fine hair such that the skin is protected from ultraviolet light and the hair is conditioned by the hair conditioner.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a front view of a hair conditioning sunscreen device according to an embodiment of the disclosure.

FIG. 2 is a side view of an embodiment of the disclosure in use.

FIG. 3 is a schematic diagram of ingredients of an embodiment of the disclosure.

FIG. 4 is a schematic diagram of a mixture of an embodiment of the disclosure.

### DESCRIPTION OF THE PREFERRED EMBODIMENT

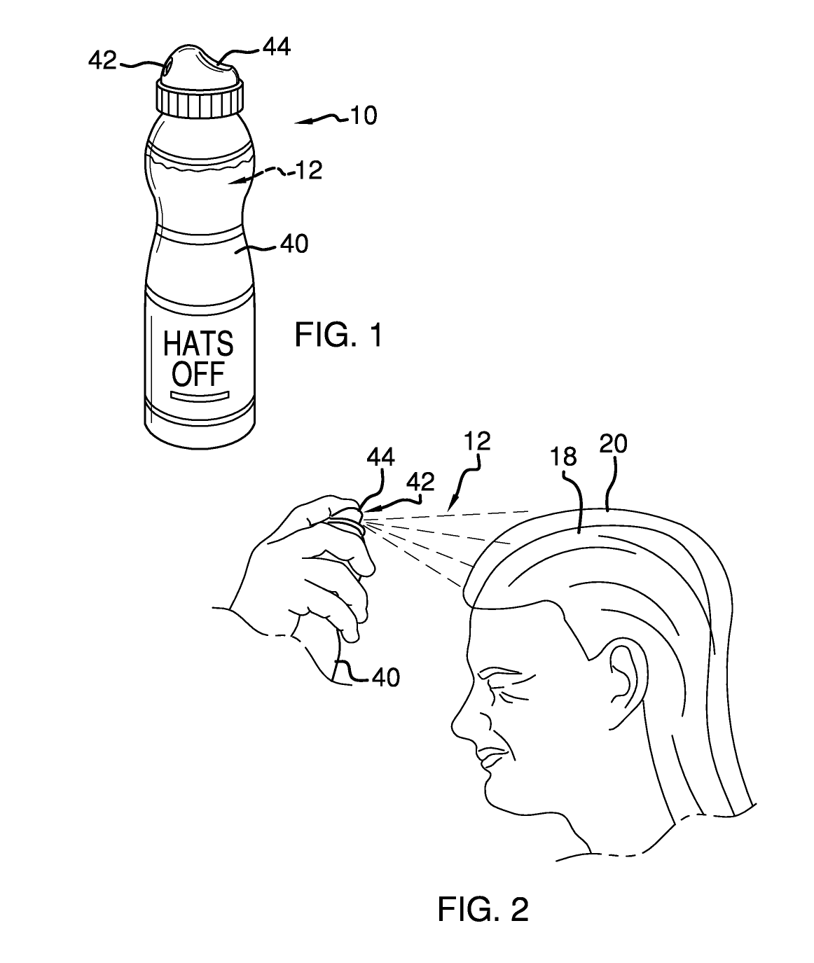

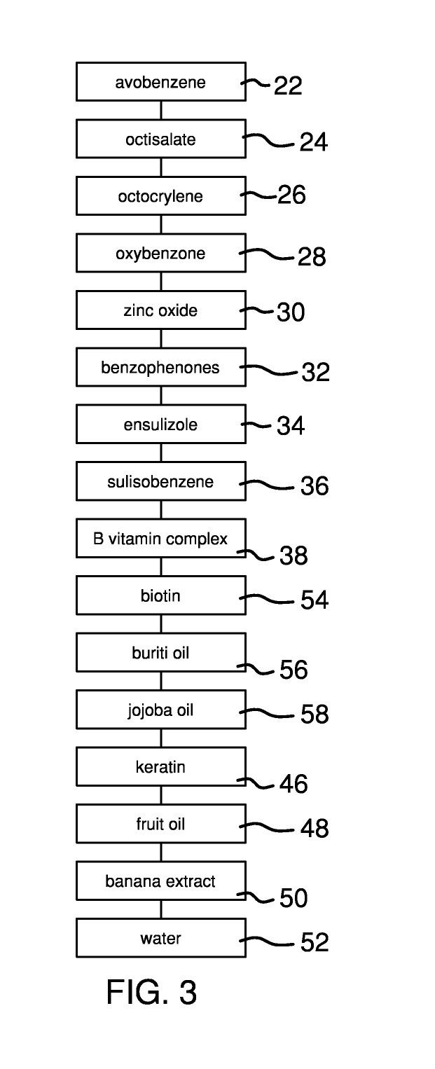

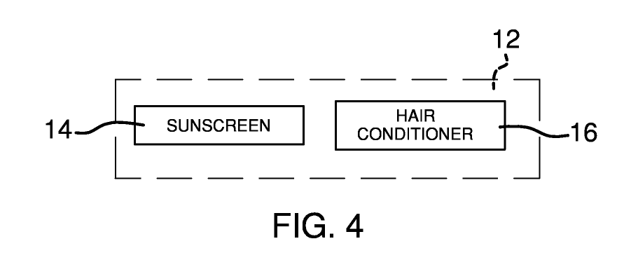

With reference now to the drawings, and in particular to FIGS. 1 through 4 thereof, a new sunscreen device embodying the principles and concepts of an embodiment of the disclosure and generally designated by the reference numeral 10 will be described.

As best illustrated in FIGS. 1 through 4, the hair conditioning sunscreen device 10 generally comprises a mixture 12 of a sunscreen 14 and a hair conditioner 16. The sunscreen 14 and hair conditioner 16 may be of conventional formulations providing the sunscreen 14 and hair conditioner 16 in fluid form. The mixture 12 is applicable to skin 18 covered by hair 20 such that the skin 18 is protected from ultraviolet light and the hair 20 is simultaneously conditioned by the hair conditioner 16 through a single application of the mixture 12. The mixture 12 comprises between three percent and nine percent by weight of avobenzene 22, between three percent and seven percent by weight of octisalate 24, between three percent and seven percent by weight of octocrylene 26, between six percent and ten percent by weight of oxybenzone 28, between three percent and seven percent by weight of zinc oxide 30, between two percent and six percent by weight of benzophenones 32, between three percent and nine percent by weight of ensulizole 34, between three percent and seven percent by weight of sulisobenzene 36, between four percent and eleven percent by weight of B vitamin complex 38, between seven percent and eleven percent by weight of biotin 54, between three percent and seven percent by weight of buriti oil 56, between three percent and seven percent by weight of jojoba oil 58, between three percent and seven percent by weight of keratin 46, between three percent and seven percent by weight of fruit oil 48, between three percent and seven percent by weight of banana extract 50, and between five percent and nine percent by weight of water 52.

A container 40 is provided. The mixture 12 is positioned in the container 40. A valve 42 of conventional design is coupled to the container 40. The mixture 12 is dispensed from the container 40 when the valve 42 is opened. A trigger mechanism 44 of conventional design is coupled to the container 40 and operationally coupled to the valve 42 such that manipulation of the trigger mechanism 44 opens the valve 42 to dispense the mixture 12. A propellant may be provided in the container 40 and the mixture 12 may be under pressure within the container 40. Alternatively, the valve 42 and trigger mechanism 44 may incorporate a pump or the like for dispensing the mixture 12 from the container 40.

In use, the mixture 12 is dispensed from the container 40 by manipulation of the trigger mechanism 44. The mixture 12 is applied directly to the skin 18 and hair 20.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. (canceled)

2. (canceled)

3. (canceled)

4. (canceled)

5. A hair conditioning sunscreen device comprising:
a mixture of a sunscreen and a hair conditioner wherein said mixture is applicable to skin covered by hair such that the skin is protected from ultraviolet light and the hair is conditioned by said hair conditioner, said mixture comprising between three percent and nine percent by weight of avobenzene, between three percent and seven percent by weight of octisalate, between three percent and seven percent by weight of octocrylene, between six percent and ten percent by weight of oxybenzone, between three percent and seven percent by weight of zinc oxide, between two percent and six percent by weight of benzophenones other than said avobenzene and said oxybenzone, between three percent and nine percent by weight of ensulizole, between three percent and seven percent by weight of sulisobenzene, between four percent and eleven percent by weight of B vitamin complex, between seven percent and eleven percent by weight of biotin, between three percent and seven percent by weight of buriti oil, between three percent and seven percent by weight of jojoba oil, between three percent and seven percent by weight of keratin, between three percent and seven percent by weight of fruit oil, between three percent and seven percent by weight of banana extract, and between five percent and nine percent by weight of water;
a container, said mixture being positioned in said container;
a valve coupled to said container, said mixture being dispensed from said container when said valve is opened; and
a trigger mechanism coupled to said container, said trigger mechanism being operationally coupled to said valve wherein manipulation of said trigger mechanism opens said valve.
